(19) United States
(12) Patent Application Publication
(10) Pub. No.: US 2026/0123975 A1
(43) Pub. Date: May 7, 2026

(54) DNA Fixing Machine

(71) Applicant: Francisco Collado, Murrieta, CA (US)

(72) Inventor: Francisco Collado, Murrieta, CA (US)

(21) Appl. No.: 19/069,127

(22) Filed: May 24, 2025

(51) Int. Cl. A61B 18/02; A61B 18/00; A61B 18/14

(52) U.S. Cl. CPC A61B 18/02; A61B 18/1492; A61B 2018/00095; A61B 2018/00404; A61B 2018/00464; A61B 2018/00577

(57) ABSTRACT

The medical field has been trying to solve medical issues by concentrating in mitigating the problems, not in solving them. The DNA Fixing Machine goes to the root of the problem, and it uses the capabilities of your body (immune system, stem cells reproduction, body controls such as temperature sensors, blood laminar flow, etc.) to fix the disease once and for all. Most of the time, it can be done in one session. The DNA Fixing Machine won't give side effects, it won't be rejected by your body and because of its ability to have strict controls, it won't affect healthy cells. It is flexible; it fit through a standard 3 feet door, and it can be installed above a 16 wheel truck to have a mobile unit to serve places where medical buildings are far away. It can last 40 years with little or no maintenance.

### FIELD OF INVENTION

The invention relates to the field of medical devices to treat diseases; in this case it will be used to cure diseases.

### BACKGROUND OF INVENTION

The medical field had been trying to solve medical problems (diseases, health issues, etc.) mostly by treating the symptoms or underlying causes of a disease rather than fixing it in a permanent way because some diseases are chronic (last for a long time) or involve complex biological processes that are difficult to fully reverse with medication alone.

The objective of the DNA Fixing Machine is to cure the diseases, not to treat them. It will use your immune system, the ability of the different stem niches to produce the missing or damaged cells inside your body and mechanical and plumbing properties of the body like laminar blood flow to cure the disease.

It is based on the idea that the best medication is your own cells and organs, because they won't cause side effects, they won't be rejected by your tissues, they won't damage healthy cells in your body, it knows what is wrong and the sequence it needs to fix it.

The DNA Fixing Machine is also a flexible machine; it will adapt to the needs and requirements of each person and the majority of the diseases will be cured in one session instead of the multiple treatments as traditional medicine does.

It takes 10 to 15 years due to all the research and testing that it needs for a new medicine to be approved and on average it costs around 2.8 billion dollars. The DNA Fixing Machine will bypass a lot of testing because it won't involve chemicals or biological agents; it is a “treatment machine”, and the time it takes for treatment machines to be approved is between 90 and 180 days.

### SUMMARY OF THE INVENTION

A single cell is considered the smallest organism. Normally there are 12 to 13 key organelles inside a human cell, the head of the pin is the size of a million human cells. A single human cell perform a billion biochemical reactions per second; these reactions are not constant events, they are changing and adapting in response to the cell's internal and external conditions; taking advantage of the capacity available in the human body (it will be also applicable animals) to fix medical problems is the main function of the DNA Fixing Machine.

We developed from a single cell (the union of the sperm and egg, called zygote); this single cell will divide and differentiate to produce all the more than 3 trillion of cells of the human body, it knows the exact location of each cell and its function, and it has memory of where it cells goes, even if they are missing or damaged.

To use the DNA Fixing Machine inside your body, it is important that this be done in a proper medical facility, under the direction of professional medical personnel and the person or animal be under anesthesia.

In normal surgery, they tried to select an anesthetic agent that will have anti-inflammatory effects because inflammation activates the immune system and they want to reduce the effects of the immune system respond after surgery to help reduce post-operative pain and swelling, leading to a more comfortable recovery for the patient and potentially reducing the need for high doses of opioids.

The DNA Fixing Machine will be the opposite, an anesthetic agent that activates the immune system is ideal because the idea of the DNA fixing machine is to cure diseases by using your immune system or chemical messengers.

The body penetration required by the DNA fixing machine will be smaller than a MSI (minimal invasive surgery, which is around 5 cm), so in some cases local anesthesia could be used.

For local anesthesia (which is around 40% of the cost of general anesthesia), the preferred option is Prilocaine (it is injected) and causes inflammatory reactions. The cost of all the different local anesthesia agents is basically the same.

For total anesthesia, isoflurane is the recommended one. It is administered by inhaling oxygen and its cost is around 15% of a typical general anti-inflammatory anesthesia agent.

All past, present and future diseases can be grouped into twelve different categories according to the DNA Fixing Machine treatments; each of the 12 different groups of diseases need a different computer program. The groups are:

### 1) Cancer

All the cancer types are due to DNA Cell mutations. In cancer these mutations could occur due to a virus, radiation, chemicals, biological agents, etc.

Cells have built-in mechanisms to repair DNA damage and fix mistakes during replication, but once a DNA mutation has become permanent the capacity of the cells to repair the mutation or return the cell to its original state is minimal. Most of the DNA mutations are considered irreversible.

Non-cancerous (benign) tumors are a difference of cancer tumors, they don't divide forever because their cells, while abnormal, remain within the normal cellular framework, and they don't invade and spread like cancerous cells. Benign tumors grow slowly, remain localized, and are encapsulated, so they are easy to remove because they have a defined shape. The DNA Fixing Machine can be used to eliminate benign tumors that are in critical areas where they could cause medical problems and it is hard to operate.

DNA mutations can influence cell volume, resulting in larger or smaller cells volume, this is typical of cancer. They alter the cell's volume by disrupting normal cellular processes that regulate size and growth.

Tumor blood vessels are structurally and functionally abnormal. They exhibit irregular diameters, branching patterns, and are often leaky. Tumor vessels are also more abundant at the tumor-host interface and tend to decrease in density as the tumor grows, leading to deprivation of oxygen and areas of dead cells inside the tumor (necrosis).

Lack of oxygen leads to hypoxia (low level of oxygen in the cells at the tumor), which promote tumor growth and metastasis by driving angiogenesis (formation of new blood vessels from existing blood vessels). This makes the cancer cells resistant to radiation therapy, chemotherapy and drug treatment due to its lack of oxygen; these treatments need good oxygenated cells to work.

The DNA Fixing Machine will bypass these problems.

Hypoxia forces cancer cells to switch to anaerobic metabolism (energy production without oxygen). Anaerobic metabolism produces only 5% of energy compared to aerobic metabolism (using oxygen, which normal healthy cells due), so you need to input a lot of more energy to produce the same result. This is one of the reasons people with cancer lose weight fast. Bear in mind that this anaerobic metabolism occurs naturally when persons do intense activities like sprinting where rapid bursts of energy are needed for short periods of time, but we are not designed to do this all the time as cancer cells force us to do.

Chemotherapy and radiation is used in cancer treatments to kill the cancer cells, slow their growth, and reduce the risk of recurrence. They are used to kill rapidly dividing cells but they also damage healthy cells with rapid growth, such as hair follicles and digestive tract cells.

Another option used these days to treat cancer is tumor removal surgery, but it has temporary and permanent effects like nerve damage, swelling, bruising, and pain. Cancer tumors don't have a well defined shape, so there is a big possibility that the tumor will return after surgery.

Medical field had been trying to kill the tumor by using extreme cold (cryoablation) and extreme heat (hyperthermia, killing cancer cells at 42 degrees Celsius). The major problems with these treatments is that the treatment is designed to keep the healthy cells intact, so the edge of the tumor where the dangerous cells are located most of the time is spare of treatment and the tumor is not killed in its totality and it will return.

The DNA Fixing Machine can be used in three different ways to kill the tumor cells.

A) Mutated DNA cells die at lower temperature compared with similar healthy cells because mutations disrupt normal cellular functions, making them more vulnerable to stress and they have a weakened cell temperature resistance. Cancer cells die at around 42 degrees Celsius compared with healthy cells that die at 44 or higher degrees Celsius. The first use of the DNA Fixing Machine is to keep the area where the cancer cells are located at 42 degrees long enough to kill all the mutated and cancer cells without damaging healthy cells. The DNA Fixing Machine can control cell temperature to a plus or minus 0.1 degree Celsius and it can also control the exact area it needs to be at that temperature. This is similar to the idea of using hyperthermia, but hyperthermia use these days can cause healthy tissue damage and other side effects if the temperature is too high. In hyperthermia, days are very difficult to control temperature precisely because of the complex interplay between heat delivery, tumor microenvironment, and the body' natural cooling mechanisms. It uses microwaves or radio frequency and they cannot be controlled easily because it is hard to match the heat in with the heat out to keep the tumor at the right temperature. The DNA Fixing Machine uses a conduction wire that is controlled by air convection, allowing perfect temperature control and it can matches the heat in with the heat out needed at the tumor to keep the correct temperature to kill all the cancer cells and it can adjust the temperature every second is needed (this is part of the computer program).

B) The DNA Fixing Machine can also return the cancer cells to its original volume by providing the needed cold or heat required. When the cell volume is bigger or smaller than 10% of its original volume, antibody-antigen interaction (the immune system responds), is disrupted altering the accessibility and conformation of the antigen on the cell surface, essentially changing the shape of the epitope (the part the antigen that the immune system recognize) that the antibody needs to bind, preventing a proper fitting and making the immune system useless in this case. This is how viruses are killed by the immune system, by killing the damaged cell.

Returning the cell to its original size will make the cancer cells visible to the immune system.

C) The DNA Fixing Machine can also be used to kill the cancer cells by cryoablation (temperatures lower than 0 degrees Celsius). A difference of the cryoablation method used these days where the cold agent is liquid nitrogen, liquid nitrous oxide, or compressed argon gas where cooling is produced by using the Joule-Thomsom effect and a high-pressure, closed-loop gas system. The Joule-Thomsom effect is a temperature change in a gas when it expands under pressure. The DNA Fixing Machine uses the peltier effect, where temperature is created when an electric current flows through a junction of two dissimilar materials. The Peltier effect offers better temperature control compared to the Joule-Thomsom effect, the Peltier effect allows for precise and rapid temperature changes by varying electric current, while the Joule-thomson effect relies on pressure changes and is less readily controllable. Peltier devices, especially with feedback circuitry can achieve high temperature stability, making them ideal for applications requiring precise temperature (cell cancer killing).

### 2) Reproduction of Neuron Cells

Neuron cells are the cells of the nervous system. They carried messages through an electrochemical process. The average human brain has approximately 86 billion neuron cells.

Neuron cells as any other cell have cell membrane, nucleus, mitochondria and other organelles. The organelle lysosomes contain digestive enzymes that break down various molecules like proteins, lipids, and carbohydrates; this organelle is very important to the neuron cells and when it is not working properly, it causes mental or brain problems.

Lysosomes are the waste disposal and recycling organelle of the cell (the garbage system). Lysosomes are controlled by the DNA (the computer system of the cell).

Defects in lysosomal function lead to the accumulation of abnormal proteins and cellular waste inside the cell (plaques), altering the cell function and eventually causing the neuron cell to die. These plaques also can also move outside the cell, affecting the communication between neuron cells contributing to the disease's progression. These plaques cause diseases like Alzheimer's and Parkinson's.

A neuron cell consists of the main body where all the organelles are located, an axon (a long “cable” that carries nerve impulse away from the neuron toward target cells; there is only on axon per neuron cell), and dendrites (numerous short branches “small cables” that conveys nerve impulses toward the neuron cell.)

The Amyloid beta plaques (typical characteristics of Alzheimer), this plaque accumulated around and between neurons, disrupt neuron normal functions, triggering a cascade of toxic reactions that lead to cell damage, inflammation, and ultimate to cell death by apoptosis (method used by the immune system to kill cells that are not working properly).

Normally, when a cell is killed by apoptosis, another healthy cell next to the one kills duplicates to fill up the “open space”. In neuron cells this is not the case because neuron cells don't have the organelle centrioles, which is critical for initiating cell division. Once a neuron cells madurez (middles 20's), it exits the cell cycle and cannot replicate itself, making it a permanently differentiated cell. if a neuron cell dies after its maturation time, its “space” cannot be filled under normal body conditions.

In Parkinson's diseases the primary protein that forms abnormal aggregates is called alpha-synuclein, which accumulates into clumps known as Lewy bodies, but the result is similar to Alzheimer, dead of neuron cells providing holes in the brain.

The treatments for dead neuron cells are mostly to treat brain injuries or diseases to manage the symptoms but neuron cell replacement is beginning to be an option.

Neuron cell replacement consists of manipulating stem cells in a lab. The problems with this treatment is that production and installation of stem cells need to follow a sequence that changes from person to person and from contention to condition, plus sometimes cells of a different person are used, so tissue rejection is a big problem.

The body is full of “prime material” to provide missing cells or to replace faulty cells in case of they are needed, they are called stem cells and they are neutral cells that will differentiated to any cell needed by the body by duplication; the original cell remains a stem cell making this ra material process an infinite cycle, and the other cell change to any cell needed by the body.

There are brain stem cells for future duplication that when activated will work to fill up these “holes” caused by missing neuron cells with the correct cell type;

Brain stem cells navigate to their proper location by responding to chemical signals from their cell environment, particularly “chemokines” (small proteins that act as chemoattractants, they guide cells to specific locations). They are released by damaged tissue or specific regions of the brain, which bind to receptors on the stem cell surface, guiding them to the necessary area where they are needed to repair or regenerate tissue; essentially, the brain provides a “homing signal” to attract stem cells to the right place.

In the brain there are two neural stem cell niches, which are specialized tissue microenvironments that regulate stem cells; when activated they control how quickly they divide and what the daughter cells need to be. These niches contain self-renewing, multipotent cells that give rise to all components of the central nervous system. Spinal cord injuries are part of the central nervous system and the stem cell homing signal also works on the spinal cord, meaning that neutral stem cells can be attracted to and migrate towards an injured area within the spinal cord due to specific chemical signals released at the injury site, making it a potential therapeutic target for all types of spinal cord injuries.

Cervical (affects the neck), thoracic (affects the upper back and chest), lumbar (affects the lower back), and sacral (affects the sacrum bone). Spinal cord injuries can cause full or partial paralysis.

Other cells in the brain, like astrocytes and microglia, can also contribute to the guidance of stem cells by releasing signaling molecules.

The DNA fixing Machine a difference of the existing neuron cell replacement when the duplication of the stem cells is done by using chemicals in a lab, it will activated the neuron stem cell niche inside the body, so it produces the correct neuron cell in the correct sequence to fill the holes done by dead neuron cells or to fix any neuron problem.

In Azheimer's disease memory loss is due to loss of communication between brain cells rather than death of cells themselves, so once the communication is established again, the memory will come back.

Besides the normal brain problems, the reproduction of neural stem cells can fix other diseases that cause dead neurons, such as the polio virus.

The neuron niche is not a single, defined size, but rather a region within the brain that varies depending on the specific niche, with the most well-known being the subventricular zone (SZC) which can be 4 millimeters deep and span a large area along the lateral ventricles of the brain, individual stem cells here range from 4 to 100 micrometers in diameter, which include all the different neuron cells needed by the brain or nervous system. This is the neuron niche the DNA fixing machine will activate; once activated the neuron niche will produce the needed cells type on the correct sequences.

The DNA fixing Machine not only can control the temperature needed to plus or minus 0.1 degree Celsius in the worst case, but is also flexible enough to change temperature right away as needed to make the cure a dynamic process; most of the treatments available are static processes.

Research has shown that neural stem cells tend to activate at slightly lower temperature than the body, around 35 degrees Celsius. This lower temperature encourages their activation and differentiation. Stem cells possess receptors like TRPV4 which are activated at the right temperature.

When neuron temperature changes, it can disrupt the balance of ions across the cell membrane, creating problems like altered firing rate, impaired signal transmission, potential for spontaneous firing, all of which can significantly impact neural function and cause neurological issues. The key to avoid this is to activate the neuron stem cells without changing the temperature of the cells next to the niche, something that the DNA Fixing Machine can easily do.

Neutral stem cells are known for their quick division rate, allowing them to generate a large number of cells relatively quickly, but the exact time that it will take to cure the disease will depend on the amount of missing neurons. While dividing rapidly, neutral stem cells can also differentiate into different types of cells like neurons, astrocytes (support cells for the neurons) and oligodendrocytes (they provide the insulating layer that wraps around the nerve fibers (axons). They can provide all the parts needed to fix any brain or spinal cord problem (missing or replacement damage neuron cells).

Lack of insulation on the axon can cause symptoms such as muscle weakness, impaired coordination, vision problems, difficulty with speech, cognitive difficulties, and sensory disturbances. The solution to these problems is the same, reproduction of neuron stem cells by the DNA fixing Machine.

There are some “brain diseases” caused by imbalance in the communication between neuron cells, such as depression, bipolar disorder (extreme mood swings), anxiety disorders, ADHD (attention deficit disorder), and schizophrenia. Their causes are not well known, although postmortem examination of schizophrenic brains showed areas of the brain with decreasing density, suggesting that missing or dead neurons could be the main reason for these diseases. If this is the problem, the DNA Fixing Machine can also work here.

### 3) Arterial Blood Vessel Maintenance

In human and other vertebrates (animals that have a backbone and a skeleton) internal transport is accomplished by a closed circulatory system, the cardiovascular system.

The components of the cardiovascular system are the heart, blood vessels and blood.

In humans the heart consists of two atria, the right atrium receives oxygen poor blood from the body and the left atrium receives oxygen rich from the lung. Also, below the atria there are two ventricles which are the pumps, the right ventricle pumps oxygen poor blood to the lungs (low pressure) and the left ventricle pumps oxygen rich blood to the body (high pressure). The pumps work in parallel and they rely on pressure differential to work.

They are natural pressure sensors, also known as baroreceptors inside the high pressure loop. These sensors detect changes in blood pressure to regulate the pumping force. The low pressure pump adjusts as required to keep the pressure difference; if the high pressure pump increases in value (systolic number), the low pressure pump will also increase in value (diastolic); which is the condition of having pumps working in parallel.

The blood vessels consist of arteries, veins and capillaries, which in the human body have been estimated to extend a total of 100,000 kilometers. Arteries carry blood away from the heart to organs throughout the body. Within the organs, arteries branch into arterioles, tiny vessels that give rise to the capillaries. Capillaries form networks of microscopic vessels that infiltrate each tissue. It is across the thin walls of the capillaries that chemicals are exchanged between the blood and the interstitial fluid surrounding the cells. At the end, the capillaries rejoin to form venules, and these small vessels converge into veins. Veins return blood to the heart.

Capillaries can be blocked, but they have a unique mechanism to clear blockages by growing a membrane around the debris, essentially encapsulating it and allowing the blood vessel to function again. Capillaries won't be a part of the blood vessel maintenance because they don't need maintenance, they fix themselves if they have a problem.

Veins return blood to the heart by utilizing one-way valves, which prevents blood from flowing backwards and combined with the squeezing action of surrounding muscles that push blood toward the heart, particularly at the legs. The vein valves are primarily located on the medium and large veins, especially at the arms and legs. Vein valves are flap-like structures that prevent blood from flowing backward. They work passively, like swinging doors, opening when blood flows past them and closing when flow slows.

When vein valves are damaged or weakened, blood can flow backwards and pool in the veins, especially on the legs. This leads to varicose veins, pain, skin discoloration.

The best solution to treat damaged valves is to close the vein. Once the vein is closed, the blood is re-routed to other healthy veins, fixing the problem.

Veins are closed by heating the inside of the veins to around 70 degrees Celsius or higher, causing the collagen in the walls to shrink and closing the vein. Going over 90 degrees Celsius could damage the cells outside the vein vessels. This treatment is called ablation (using heat).

In vein ablation, the material of the catheter tip electrode that delivers the heat from radiofrequency energy is made of Dacron, polyurethane, platinum-iridium, diamond or nitinol.

Dacron has a thermal conductivity of 0.24 W/m K, polyurethane has a thermal conductivity of 0.03 W/ m K, platinum-iridium has a thermal conductivity of 30 W/mk, diamond has a thermal conductivity of 1,500 W/mK and nitinol a thermal conductivity of 20 W/mK. All the above electrodes but nitinol are designed for single use. The higher the thermal conductivity the more temperature control you can have to a certain point. Low thermal conductivity can hinder efficient heat dissipation, causing overheating problems. Very high conductivity can lead to unwanted heat transfer.

Vein ablation normally includes cooling for the reasons above; the materials are very low thermal conductivity or too high thermal conductivity.

For example, diamond tip catheters require a cooling system (saline irrigation) to prevent char and thrombus due to the high heat transfer of the material, its average price is $2,000 per piece and it can only be used once.

The DNA Fixing Machine can be used to treat veins with ablation, it uses silver medical grade that has a thermal conductivity of 429 W/mk (big enough to provide good temperature control, so no additional cooling is required), and it can be reused hundreds of times after cleaning. It also allows you to adjust the length of the part that needs to get inside the vein.

The biggest problem in the blood vessels is the formation of plaque at the artery walls. These plaques can cause major health problems.

Plaques can cause blood Clots When you get a cut, blood cells called platelets plug the hole in the damaged blood vessel with a clot (a thickened liquid) that stops bleeding. Sometimes these plaques rupture, exposing the underlying artery wall to the blood. This triggers a series of events that leads to clot formation, narrowing the artery and obstructing blood flow.

Clots are dangerous because besides preventing organs of vital oxygen, they can break off and travel in the blood to the lungs causing pulmonary embolism, they can block blood to the heart causing a heart attack, a block in the brain can cause a stroke, etc.

The plaques are covered with a fibrous cap, a barrier between the paque and the blood vessel lumen (open space where blood moves), protecting the artery from bits of plaque breaking off and causing a clot.

It takes decades for a plaque to reach a point where it significantly narrows the arteries and causes symptoms.

Right now the standard way to treat blood vessel plaques is by a combination of lifestyle changes, medications and in some more dangerous cases procedures like angioplasty, which is an invasive product that widens narrowed or blocked arteries. The procedure typically involves inserting a catheter with a balloon at the tip, which is inflated to open up the narrowed artery.

A small mesh tube called a stem may be inserted to help keep the artery open. There is also laser angioplasty but it is very expensive and it is not as effective as balloon angioplasty, so it is not used often. In the worst case, bypass surgery is the standard solution to remove plaques.

Once a plaque is formed, it is difficult to revere. They form in areas of disturbed flow, not where flow is laminar.

To recover from bypass surgery takes between 1.5 to 2 months on average.

One of the major problems with angioplasty is that the catheter will hit the plaque with the possibility that part of the plaque will get loose, causing a clot to move in the blood vessels.

Adding laminar flow to a blood vessel containing plaque will help to stabilize the plaque and reverse the plaque's negative effects. It will take 30 days of constant laminar flow to reduce and start eliminating the plaque.

Laminar flow is a smooth orderly flow where fluid particles move in parallel layers with minimal mixing. It is low velocity.

Transitional flow is the intermediate state between laminar and turbulent. It has an unstable flow with characteristics of both, laminar and turbulent.

Turbulent flow is a chaotic, irregular fluid motion with swirling eddies. High velocity with significant mixing of layers and hard to predict.

The flow of blood inside the arteries is laminar; full laminar is good for the blood vessels because it promotes smooth, orderly movement of blood through the vessels, minimizing energy loss and preventing damage to the blood vessel walls. The shear stress (force exerted on the vessel wall by blood) is relatively low and evenly distributed, reducing the damage to the endothelial lining of the vessel.

Very slow laminar flow is considered bad for the blood vessel walls because it can lead to stagnant (no movement) blood, which increases the risk of platelet aggregation, clot formation, and damage to the endothelial lining vessel, particularly in areas with low shear stress allowing plaques to form. The areas of low shear stress in a blood vessel are at the inner curvature of bends, at the branching point of arteries, and immediately downstream from a stenosis (narrowing). They are considered the fittings of the blood vessels.

Transitional flow is not considered good for the blood vessels because it has varying shear stress patterns, which can activate inflammatory responses within the arterial wall promoting plaque formation. Transitional flow often occurs near bifurcations (division into two or more parts or fittings) or around existing plaques.

Turbulent flow is bad for the blood vessel walls because it creates chaotic shear stress on the endothelial lining, disrupting its normal function and promoting the development of vascular diseases like atherosclerosis, where the irregular forces can damage the vessel wall, leading to plaque formation, inflammation, and potential rupture of the vessel.

In blood flow a Reynolds number of less than 2,000 is considered a laminar flow and above 3,000 is considered a turbulent flow. A Reynolds number between 2,000 and 3,000 is considered a transition fluid.

Let's look at some Reynolds number calculations for the blood flow.

The average velocity of blood in the arteries, particularly in the aorta (the main artery leaving the heart) is 0.3 meters per second at rest; the heart has the capacity to increase the velocity at the aorta to over 1 meter per second.

The blood pressure at the aorta is 120 mmHg under normal conditions, a number bigger than 130 mmHg is an indication of high blood pressure.

The blood pressure at the last baroreceptors is around 60 mmHg under normal conditions.

The average exterior diameter of the main aorta is 0.0281 meters. The aorta wall is 0.003 meters thick for a total of 0.006 meters of wall. The inside diameter of the aorta is 0.0221 meters.

The Reynolds number is giving by the formula:

$$Re = De * V * D / ( FV )$$

Where:

Re is the Reynolds number (a dimensionless number)

De is the fluid density in Kg/(m\hat{ }3). The flood density of blood is 1,050 Kg/(m\hat{ }3) at 37 degrees Celsius.

Blood velocity changes with age, gender and hematocrit (percentages of blood cells). In females the blood velocity is between 3% 5% higher than males. Let's use the average velocity of 0.3 meters per second.

FV is the blood dynamic viscosity in Kg/(m*sec). Blood fluid viscosity decreases as temperature increases. The blood dynamic viscosity at 37 C is 0.0035 Kg/m-sec.

The Reynolds number at the aorta coming out of the heart is:

$$Re = ( 1 , 050 * 0.3 * 0.0221 ) / 0.0035 = 1 , 989$$

It is a fully developed laminar flow.

During exercise, the body temperature can go up all the way to 40 degrees Celsius (blood vessels start to get damaged if the blood temperature goes to around 50 degrees Celsius).

Let's do another Reynods number calculation to see what kind of blood flow will occur with heavy exercise (blood temperature at 40 degrees Celsius), blood flow at 1 m/s and with the blood vessel opening increase of 20% (typical for exercises). The diameter of the blood vessel will be 0.02421 meters.

The De now will be 1,044 Kg/(m\hat{ }3) (Drop 2 kg/m\hat{ }3 per degree C.).

The FV now will be 0.00329 Kg/m-sec. (Drop 2% per degree C.).

$$R e = ( 1 , 044 * 1 * 0.02421 ) / 0.00329 = 7 , 682$$

This is a turbulent flow; it means that the human blood vessels are designed to handle turbulent floors for a while without any problems. Studies have shown that increased turbulent flow for a while is good for the blood vessels because the increased shear stress stimulates the lining of the blood vessels to promote healthy function and adaptation.

As the first set of calculations showed, the average blood coming out of the heart is laminar under normal conditions and as it moves through the arteries, its Reynolds number is reduced more. Looking at the Aorta Arch location (the inside diameter of the blood vessel here is 19 mm). The calculated Reynolds number is below.

$$R e = ( 1 , 050 * 0.3 * 0.019 ) / 0.0035 = 1 , 710$$

The blood flow is still laminar, but let's see what happens if the blood vessel splits into two.

Blood vessels split following the Murray's law, which is:

$$( D 1 \hat{} 2.7 ) = ( D 2 \hat{} 2.7 ) + ( D 3 \hat{} 2.7 )$$

For calculation purposes, let D2 be equal to D3.

$$( 0.019 \hat{} 2.7 ) = 2 * ( D 2 \hat{} 2.7 ) so$$

$$D 2 = 0.0147$$

The flow volume in a pipe is Q=V*A (velocity times area). This formula can be used to find the velocity at the branches (because they have the same diameter, they wll have the same velocity).

$$Q 1 = Q 2 + Q 3$$

$$( 0.3 ) * ( Pi ) * ( 0.0095 \hat{} 2 ) = 2 * ( V 2 ) * ( Pi ) * ( 0.00735 \hat{} 2 )$$

$$V 2 = 0.2 \; 506 meter / second$$

The Reynolds number at the branches will be:

$$R e = ( 1 , 050 * 0.2506 * 0.0147 ) / 0.0035 = 1 , 105$$

A Reynolds number less than 1,000 will be a problem because at this low number flow separation at the wall could occur because abrupt changes in geometry at low Reynolds number causes the fluid to detach from the blood vessel wall and forms eddies or recirculation zones. These eddies after a while will damage the walls of the blood vessels and plaques will form.

If the above calculated number is reduced (1,105) to less than 1,000, and they are many reasons for this, such as high cholesterol levels in the blood, smoking, diet (eating foods with saturated and trans fats), lack of exercise, age (cholesterol levels increased with age), genetics, alcohol, etc., in a continuous base, a plaque will form.

Plaque buildup, or fatty deposits, in the arteries are called atherosclerosis. These deposits are made up of cholesterol, fatty substances, cellular waste products, calcium and fibrin, a clotting material in the blood. This buildup narrows or blocks arteries, reducing blood flow and the amount of oxygen and nutrients reaching the body.

As the diameter of the blood vessel gets smaller and smaller due to plaque accumulation, the flow won't change and this means that the velocity increases (see Q1=Q2 calculation below), causing the Reynolds number to get bigger and bigger until the flow becomes turbulent and this will be an issue with the plaque cap, because it will be destroyed after awhile and clots will move inside the blood flow.

Let's see how much it will take for the Aorta Arch to become turbulent.

Assuming that the plague will block 44% of the diameter of the blood vessel and using the formula:

$$Q 1 = Q 2$$

$$V 1 * ( Pi ( R 1 \hat{} 2 ) ) = V 2 * ( Pi * ( ( 0.56 * R 1 ) \hat{} 2 ) )$$

$$V 2 = 3.189 V 1$$

Let's use this number on the calculated value of the blood after the Aorta Arch to see how the Reynolds number changed.

$$R e = ( 1 , 050 * 0.9566 ⋆ 0.0100 \; 64 ) / 0.0035 = 3 , 053$$

This Reynolds number is a turbulent flow, so once an artery is reduced by around 44%, the flow changes to turbulent at that location, and there is a good change that the plaque cap will be destroyed and a piece of the plaque will break and clot will be circulating in the blood looking for a place to cause damage.

Medical science uses this turbulent flow to know where the plaque is located. Also, the plaque at the wall will have higher temperature because of the reduction in conduction at the wall, the thicker the plaque the less conduction will occur and the higher the temperature at the plaque/blood vessel wall. This number is used by the medical field to know if a piece of the plaque will break and a clot will form.

The DNA Fixing Machine can return the blood flow to laminar at the plaque locations by heating the blood locally and causing the arteries to dilate (vasodilation). Arteries can dilated up to 50% of its original value, which is bigger than the 44% calculated above to return the flow to laminar.

One option is to keep the patient connected to the DNA Fixing machine for 30 days (not very logical); the other option is heat up the blood locally with the DNA Fixing Machine and introduce a stent (a small, wire mesh tube) during the treatment when the blood vessels are dilate naturally; this will return the blood flow to laminar and it will remain this way eliminating the plaque after a few months. The blood vessel will return to its original healthy state with no side effects or problems.

A difference of the treatments these days, the DNA Fixing Machine can return the blood flow to laminar without any risk of causing a clot.

Plaques also are the cause of high blood pressure. It occurs when the force of blood against the artery walls is consistently too high. Hydrostatic pressure is the force that moves fluids through pipes; the hydrostatic force that blood exerts against the wall of a vessel is called blood pressure. High blood pressure kills over 670,000 persons a year in the USA and over 7.5 million a year in the world.

The body constantly monitors blood pressure through receptors in the arteries, sending information back to the brain which then adjusts the heart rate and blood vessels diameter to maintain appropriate pressure in the system.

The artery blood pressure receptors (baroreceptors) are located in the carotid sinus (at the division of the carotid artery) and the aortic arch. If the arterial blockage occurs after the aortic arch (last pressure sensor), it could cause eye diseases, stroke or problems on the brain because the heart cannot adjust the blood flow depriving these cells of oxygen. If the plaque occurs before the last pressure sensor, it will cause the heart to pump harder to keep the right pressure at that location, this will cause diseases like high blood pressure and if the heart works too much the pump will overheat and it will fail causing a heart attack.

When the arteries are partially blocked, they increase the resistance to blood flow and the heart needs to pump harder to exert more force to push blood through the narrowed and less flexible arteries, causing high blood pressure and other diseases.

Blood pressure and blood vessel diameter follows the poiseuille's law, which is:

$$P = ( 8 * ( Ks ) * L * V 1 ) / ( R 1 \hat{} 2 )$$

Eliminating the constant Ks (dynamic viscosity) and L (length of pipe) because they will be the same, for the original pressure drop (P) without plaque we have:

$$P = 8 V 1 / R 1$$

Using the 44% diameter reduction above due to the plaque, we have:

$$P 2 = 8 * ( 3.189 V 1 ) / ( 0.36 R 1 ) = 71 V 1 / R 1$$

In the case with the plaque, the pressure drop increased by 8.875 times and this assuming the plaque length didn't increase. The pressure drops of the plaques accumulate, even if they are not thick enough to cause turbulent flow, but having more than one will add pressure drop to the system causing the baroreceptors to increase blood pressure by pumping harder; the result will be high blood pressure.

The opposite of high blood pressure is low blood pressure or hypotension. Low blood pressure usually is not a problem, it can be fixed by eating well, staying hydrated, etc. The exception is when it is caused by a defective heart. When blood pressure falls, the body senses a potential lack of oxygen and nutrients reaching vital organs and to counteract this, the nervous system triggers an increase in heart rate (tachycardia) to pump more blood and oxygen around the body more quickly.

Fixing a damaged heart is part of the stem cell reproduction process.

### 4) Replication of Epithelial Stem Cells—Thymus Organ

All the major organs have the capability of producing replacement for damaged or missing cells using stem cells and the thymus organ is not the exception.

There are over 100 known autoimmune diseases, with diabetics type I and Lupus being two of the most known and studied autoimmune diseases.

Some autoimmune diseases are expensive to treat, for example multiple Sclerosis costs around $60,000 a year per person just in medication (not including hospitalization or long term treatment). Lupus can easily cost $70,000 per person in medications and diabetes costs over $450 billion a year in the USA.

An autoimmune disease occurs when the body's immune system mistakenly attacks its own healthy tissues and organs.

In the case of Lupus, it leads to inflammation and damage to various parts of the body, including the joints, skin, kidneys, heart and brain.

In the case of diabetics type I; when your blood sugar level rises (after eating for example), the beta cells in the pancreas release insulin which signals cells, particularly in the liver, muscle, and fat tissue, to take up glucose from the bloodstream. Once inside the cells, glucose can be used for immediate energy or stored as glycogen in the liver and muscle for later use. Insulin promotes their storage process.

In diabetics type I, the immune systems kill a good portion of the beta cells, disrupting the production of insulin and the storage process. More glucose remains in the blood, leading to a buildup of glucose in the bloodstream. Over time this high levels of glucose will damage blood vessels and nerves that lead to complications such as kidney damage (kidney failure in some cases), eye damage (vision loss and even blindness), nerve damage (pain, numbness in feet and hands), it can cause blood plaques that lead to heart problem, and a lot of other organ failures.

Autoreactive T-cells are immune cells that mistakenly recognize and attack the body's own tissue and organs.

During the development of the T Cells in the thymus, a process called negative selection, developing T or B lymphocytes that will react with the body's own tissues are eliminated. However, during this process, some autoreactive T cells (cells that attack their own tissue and organs) escape this process and remain in the body; once they find their target antigens, they become activated and release inflammatory mediators that kill the cells; creating an autoimmune disease.

The main problem with autoimmune diseases is not the killing of the healthy cells because the body is designed to reproduce dead cells except in cases where the cells cannot reproduce like nerve cells, but it is the clearance, processing and the inability to reproduce the dead cells fast enough.

Healthy cells naturally undergo programmed cell death (apoptosis) and are efficiently cleared by phagocytic cells (like macrophages). These phagocytes engulf the dead cells, digest their contents, and prevent the release of intracellular components that could trigger an immune response.

In autoimmune diseases this doesn't happen. Sometimes the immune system fails to eliminate these autoreactive T-cells that continue to attack their own tissues or organs. In other cases, the dead cells are not eliminated fast enough causing the release of cell components which the immune system recognizes as foreign killing healthy cells, in other cases the autoimmune disease increases the cell death rate. Our system is designed to remove dead cells not in big amounts and in a continuous process as it occurs with autoimmune diseases.

The most used treatment these days for autoimmune diseases is to suppress the immune system to reduce inflammation and organ damage. This often involves medications like corticosteroids, immunosuppressants, and targeted therapies.

The problem with autoimmune medications, first is that they are a treatment, not a solution and second is that they are side effects, limiting its use and effectiveness. Most of the treatments weaken the immune system, increasing the risk of infections, others cause high blood pressure, diabetics and high cholesterol. Some medications won't work as expected because with time the body develops antibodies against the drug.

The DNA Fixing Machine will go to the root of the problem to fix it.

The medullary thymic epithelial cells (mTECs), in the thymus are the scanners of the body's tissues, specially for self-antigens. They express a wide array of tissue-specific antigens (TSAs) to test developing T cells and identify those that might be self-reactive, causing the destruction of any T cells that strongly react to self-antigens, effectively preventing the body from attacking its own tissues; this is the key to maintain immune tolerance and to avoid autoimmune diseases.

Each mTEC cell can express a broad range of tissue restricted antigens (more than 750), mTEC cells know which antigens to express through the selection of a transcription factor called Aire (autoimmune regulator), which randomly activates the expression of a wide range of tissue-restricted antigens (TRAs) from different tissues throughout the body.

Aire protein binds to the DNA of the mTECs nucleus and acts as a master regulator to switch on the expression of various tissue-specific genes. Reduction of mTECs is the main cause of autoimmune diseases, when the amount of mTECs is reduced, the central tolerance is compromised, and autoreactive T cells are less likely to be eliminated. As a result, the immune system becomes more prone to attacking the body's own tissues, leading to the development of autoimmune diseases.

The thymus is a gland located in the front of the chest. There are two niche locations of stem cells in the thymus, one underneath the organ capsule or outer layer and the other around blood vessels in the medulla, the central part. These stem cells called Poly Keratin can provide all the types of cells found on the thymus; they can be used when activated to reproduce the missing mTEC cells to return the scanner to its original healthy condition.

As in the other cases with reproduction of stem cells inside the body, the reproduction of stem cells will be directed by the messages of the body and the right cell types in the correct order needed. The thymus' activity is tightly regulated in adults, providing enough immune support to fight infections but it doesn't have extra capacity to correct autoimmune diseases. The DNA Fixing Machine can start and keep the reproduction of the mTEC cells to fix the case of the autoimmune diseases.

A thymus stem cell niche will be around 5 centimeters in diameter. The stem cells are activated at a temperature between 39 degrees Celsius and 42 degrees Celsius by a heat shock (sudden increase in temperature).

A difference of the immune system responds where the activation works as a soft start motor (starts slow and increases little by little), stem cell reproduction works as a hard motor, where they need a thermodynamic shock to get activated or a sudden increase of temperature at the beginning. The DNA Fixing Machine can do both by connecting wire #1 (the removable part), to wire #2 at the right moment. In the case of the immune system response, both wires are connected when the wire is inserted at the patient, the temperature will increase or decrease in a continuous by standard way (soft start motor).

In the case of the stem cell reproduction, wire number #1 will be introduced into the patient, the machine will operated and when the temperature is the correct one at wire #2, both wires will be connected, creating a thermodynamic shock to activate the stem cells reproduction.

The time it takes for the heat to travel in seconds from the wire #1/Wire #2 connection of to the uninsulated part of wire #1, which is the part inside the cells that will treated can be found by the below equation:

$$t = ( L \hat{} 2 ) / 2 * K$$

L is the length of the insulated part f wire #1 in meters (0.5−0.03=0.47 meters)

K is the conductivity of wire #1 (429 W/m C)

$$t = ( ( 0 \text{.47} / ( 2 * 429 ) = 0.0 \; 00548 seconds$$

The travel is instantaneous, so by connecting wire #1 to wire #2 when the correct of wire #2 is obtained, a thermodynamic shock will occur to activate the stem cells production; once the stem cells are activated, you just need to keep at the right temperature to keep the production of the stem cells and the DNA Fixing Machine can easily do this.

### 5) Removing Visceral Fat Cells

There are two types of fat, the visceral fat found deep within the abdominal cavity, surrounding organs like the liver and intestines and subcutaneous fat located just beneath the skin, which is the fat you can pinch.

Subcutaneous fat is considered good fat found on the abdomen, upper arms, legs, and buttocks. Their main functions are energy storage, it provides a readily available source of energy for the body; it works as insulation by regulating body temperature by trapping heat, and by protection, it cushions and protects muscles and bones from injury.

When you engage in exercises like aerobic, dancing, running, swimming, etc. the subcutaneous fat is the one that gets used first. The visceral fat is an additional saturated fat to be used in cases of emergency (for example lack of food), to keep the body working in good condition. This is the reason why a person with diabetes for example, is asked to get into a strict diet to control fat in the blood, he/she will lose a lot of weight because the subcutaneous fat is consumed first before any visceral fat is used, which is the one you want to remove in diabetes and other diseases because it is the bad fat. The DNA Fixing Machine will change all this, it will go directly to remove the visceral fat without affecting the subcutaneous fat.

Fat is stored in cells, an infant will have around 6 billion of fat cells, an adult around 25-30 billion and an overweight person could have between 250 to 300 billion of fat cells. Around 10% of a person's fat is visceral fat.

Visceral fat protects your organs and if affects your body functions. It is healthy and normal to have some visceral fat, but having too much isn't good, it causes many health problems, such as diabetes, heat diseases, stroke, etc.

This is the fat that goes along with having a bigger belly.

In diabetics Type II, visceral fat is associated with the lack of insulin. Visceral fat increases the amount of free fatty acids (FFAs) in the body, which lead to insulin resistance in nearby tissues. Insulin resistance is the main factor in the development of diabetics type II.

In the case of heart failure, visceral fat can accumulate around the heart causing heart failure by changing the heart structure and function.

In liver disease, excess visceral fat accumulation around the liver can lead to serious problems like cirrhosis (damage of the liver) and finally to liver failure which is fatal.

Visceral fat also makes some diseases worse. A good example is asthma. Visceral fat produces inflammatory substances that affect the airways, causing inflammation and increasing airway reactivity, essentially making it harder to breathe. This is primarily due to the release of pro-inflammatory chemicals called adipokines from the fat tissue surrounding organs like the lungs, contributing to the development and worsening of asthma symptoms. Studies show that obesity is associated with developing asthma, worsening existing symptoms, and poor asthma controls, leading to increased medication use and hospitalization. Obese patients may also experience more severe asthma, including frequent exacerbation and a higher risk of near-fatal asthma events.

The above comments are also applicable to allergies, visceral fat doesn't cause them but it helps in the development of allergies and it makes the reaction worse.

In gallbladder disease, visceral fat increases the production of cholesterol in the liver, leading to bile (a fluid originated in the liver and stored in the gallbladder, it is used in digestion of fats) saturation with cholesterol lead to the formation of gallstones.

In gout, inflammatory arthritis is caused by the accumulation of uric acid crystals in the joins due to visceral fat. Gout can lead to disability.

Visceral fat also can cause fertility problems. Research indicates that a high amount of visceral fat is linked to fertility problems in both men and women, primarily to its impact on hormone regulation and insulin resistance, which disrupt ovulation and sperm production; essentially excess visceral fat increases the risk of infertility.

Also, research had found that visceral fat can significantly contribute to erectile dysfunction (ED) due to its impact on hormones, blood vessels, and overall metabolic health, making it a key risk factor for developing the condition; essentially, a high amount of visceral fat can indirectly lead to issues with achieving and maintaining an erection.

In lower back pain, excess visceral fat is linked to lower back pain due to the added weight and altered posture it creates, causing the pelvis (it supports the upper body weight) to tilt forward and putting strain on the spine, muscles and ligaments in the lower back; essentially a large belly can contribute to a curve in the spine leading to discomfort and pain.

In osteoarthritis, a degenerative bone disease that mostly affects knees, hips and hands. It is caused by visceral fat due to its inflammatory properties which damage joint cartilage (supporting connective tissue) when present in excess. It affects over 30 million of Americans, 70% of the persons over 60 have this disease and it costs 137 billion of dollars a year in the USA.

These are just some of the diseases or problems caused by visceral fat.

Fat cells can increase 4 times their original size to store fat, a process called lipogenesis. When a fat cell reaches its maximum size, it can no longer effectively store additional fat, leading to the creation of new fat cells (a process called hyperplasia) or the accumulation of fat in organs. The body struggles to manage excess energy when the fat cells are at full capacity. Also, large overfilled fat cells release inflammatory substances, contributing to chronic inflammation (ongoing fatigue, digestive issues, headaches, brain fog (confusion, difficulty concentrating), and persistent aches and pains) associated with obesity.

Cryolipolysis also known as fat freezing is a process used to target fat cells, when they are exposed to extreme cold temperatures, they crystallize and die off, leading to the body's immune system naturally eliminating these dead cells through the lymphatic system over time resulting in a reduction of fat cells in the treated area; the fat cells are destroyed and gradually removed from the body.

Cryolipolysis is also called CoolSculpting because this system is used on areas like the stomach, thighs, hips, double chin, underarms, waist, etc.

In Cryolipolysis the layer of fat is gradually cooled between −7 degrees Celsius and −11 degrees Celsius for around one hour.

Cryolipolysis is done first by using an applicator outside the body that initiates a vacuum creating a negative pressure suction, drawing the adipose tissue closer to the surface. As the applicator brings the adipose tissue closer to the surface, it starts the cooling process killing the fat cells. The immune system will remove them.

Cryolipolysis done these days won't produce any problems, but the main issue is that because the freezing starts at the skin layer outside the body; it's not as effective as needed in the majority of the cases.

The DNA Fixing Machine can be used as a another Cryolipolysis treatment, it can easily reach the needed cold temperatures required and because it uses a wire inside the body at the fat cells location, it is more effective and you can control the freezing area and even adjusted the treated area during treatment to kill the required fat cells. The DNA Fixing Machine can kill bigger and faster areas of fat cells in one treatment.

Silver freezes at minus 961 degrees Celsius, so providing the fat cells with −11 degrees Celsius is not a problem for the DNA Fixing Machine and its parts.

Fat freezing didn't damage healthy cells because fat cells are more sensitive to cold temperatures than other cell types like skin cells or muscle cells; anyway the DNA Fixing Machine uses localcooling, so the cold temperatures won't affect the healthy cells next to the treatment area.

### 6) Tissue and Organ Regeneration

Pluripotent stem cells are cells that have the capacity to self-renew by dividing and to develop into the three primary germ cells of the early embryo and therefore into all cells of the adult body. These cells are the same as the embryonic stem cells (found on the placenta).

The three primary germ cells are the foundational blueprints that give rise to all the tissues and organs in your body. They are distinct layers of cells that emerge during the early stages of embryonic development, each with a specific destiny to fulfill. These layers are the ectoderm, mesoderm, and endoderm.

The ectoderm, the outermost layer is responsible for forming the nervous system, skin, and various components of the sensory organs. The mesoderm, sandwiched in the middle, gives rise to muscles, bone, blood vessels, and most of the circulatory (heart included) and reproductive system. Finally, the endoderm, the innermost layer, builds the digestive and respiratory system, as well as organs like the liver and pancreas.

Activated pluripotent stem cells don't know which organ or tissue is missing, instead their differentiation into specific cell types is guided by signals from the surrounding environment, including chemical clues, growth factors, and interactions with neighboring cells, which vary depending on the body's needs and interactions where cells are situated, essentially directing them to become the cell type most needed to repair a damaged or missing organ. They move inside the circulation system to the correct needed location. By using your own system to produce the needed cells; they are produced in the correct sequence to fix the problem, just a baby develops inside her mother's womb.

Pluripotent stem cells “read” signals like proteins, hormones, and other molecules present in the area, which will guide their pathway towards the appropriate cell type. Once the pluripotent stem cells are activated, they read different inputs at the same time and they decide which is the most important cell needed first and the order in how they need to be differentiated.

If a pluripotent stem cells is activated but not needed by the body (same process for stem cells at other organs), it will typically undergo a process called ‘apoptosis” or programmed cell death, essentially self-destructing to prevent unnecessary cell proliferation and potential tumor formation; however, in some cases it may remain in a quiescent state, ready to be activated if required later, depending on the specific signaling clues present in the body's environment; this means that using the DNA Fixing Machine for more time than what is needed is not an issue.

If a specific cell organ is needed and the organ has it own stem cells niche; it is recommended to active the organ stem cells first, instead of the pluripotent stem cells because when the pluripotent stem cells are activated; they will be differentiated in what body consider is the first priority, not in what an specific organ needs.

As similar stem cells for organ maintenance, they are activated by a heat shock (sudden increase of temperature to 39 degrees Celsius, but not bigger than 42 degrees Celsius).

Cells on the bone marrow (where the pluripotent cells are found), began to die at temperatures around 48 degrees Celsius when exposed to sustained period of time, so by keeping the pluripotent cells at no more than 42 degrees Celsius, no damage to any healthy cell or tissue will occur.

The bone marrow contains a large number of cells, but only a tiny fraction of them are pluripotent stem cells, its size is only a few inches in diameter. The niche is not just a space, but in the other stem cell niches, it is a specific microenvironment with specialized cells and signal molecules that actively maintain the stem cell's pluripotent state and regulate their proliferation. They are called “multipotent adult progenitor cells”, which is the equivalent of the pluripotent cells of the embryonic (embryo) stem cells.

These pluripotent stem cells are located within the stromal tissue of the bone marrow, which is a network of non-hematopoietic cells that provide structural support and a microenvironment necessary for the production of blood cells (hematopoiesis).

The primary function of the bone marrow is to produce blood cells, and an overgrowth of non-hematopoietic cells disrupts this process which leads to a disease called aplastic anemia, symptoms include fatigue, pale skin, shortness of breath, easy bruising and frequent infections due to low blood cell counts. Careful must be used to activate only the pluripotent stem cells and not the non-hematopoietic cells next to it (that is another reason to use the DNA Fixing Machine because it can easily control the activated area dimensions and the correct temperature without affecting the cells next to it.

Leukemia is the opposite issue, the overproduction of abnormal white cells on the bone marrow (hematopoietic cells). This production can crowd out normal blood cells and impair the bone marrow's ability to produce red blood cells and platelets.

In leukemia, a large amount of abnormal blood cells are produced because the bone marrow develops a genetic mutation that causes the blood forming stem cells to rapidly produce immature, dysfunctional white cells which can't properly fight infection, leading to an overproduction of these abnormal cells and crowding out healthy blood vessels. Leukemia is treated by the stem cell transplant from the patient or for some donor. Activating the patient's stem cell niche will work better than an autologous stem cell transplant because the stem cells are differentiated inside the body following the instructions needed by the tissue.

Aplastic anemia can also be fixed by activation of the body stem cells. Reproduction of stem cells is not only to replace missing cells or organs, it is also to fix damaged cells that do not function correctly.

The medical way to induce pluripotent stem production is by using techniques like induced pluripotent stem cell (reprogram adult cells back into a pluripotent state), stem cell transplantation (a bone marrow transplant), for missing limbs the medical solution these days is prosthetic limbs, the medical solution these days for organs that don't function correctly are medications, supportive care, transplantation of tissue engineering.

Salamancas have a remarkable ability to regenerate lost body parts, while humans don't. Humans have evolved to prioritize other adaptations, like strong immune systems, which may come at the cost of regeneration. The ability in limb regeneration was present in our ancestors and has been lost over time, but ability is there, it just needs to be activated.

In salamanders, a blastema, a specialized cell mass, forms at the wound site. This blastema is composed of differentiated or activated cells. Regeneration is triggered by local cues from the wound, including nerve signals and inflammatory responses; the signal tells the cells in the blastema what to do.

In humans scar tissue is a dense network of collagen and other extracellular matrix components that fills the gap where tissue has been damaged. This tissue is primarily a repair mechanism rather than a regenerative one, meaning it aims to seal the wound and prevent further damage rather than restoring the original tissue structure.

Scar tissue inhibits regeneration by blocking the formation of a blastema, a crucial cell mass required for regeneration.

The DNA Fixing Machine will be used first to remove the scar tissue by controlled heat so blastema formation will occur. The next step will be to activate the pluripotent stem cells, so communication between the blastema and the pluripotent niche is activated to start limb regeneration.

Blastema communicates with pluripotent cells through a combination of factors, including nerve signaling, wound epidermis signaling, and the release of growth factors like Fibroblast-Growth Factor (FGF). These signaling pathways initiate and maintain the differentiation process, allowing mature cells to revert to a progenitor-like state, ultimately adding differentiated or activate cells to the blastema and from there start the regeneration process.

### 7) Eye Diseases Cure

One of the most common and problematic eye diseases is caused by high eye pressure, also known as ocular hypertension, a condition where the pressure inside the eye (intraocular pressure) is abnormally high. This can damage the optic nerve, which is responsible for vision.

The eye produces a clear fluid called aqueous humor, which nourishes the eye. If the drainage system is blocked or not working properly, fluid accumulates, it increases pressure in the eye and finally causes glaucoma and damages the optic eye.

The cost of glaucoma in the USA is 2.86 billion dollars a year and it affects over 4.22 million Americans.

The drainage system is the trabecular meshwork, a spongy tissue in the eye that filters fluid out of the eye and regulates intraocular pressure. It is located in the drainage angle of the eye, between the cornea and the iris. The drainage angle is the area that gets blocked causing increased eye pressure.

There are stem cells that have the potential to regenerate a damaged trabecular meshwork, offering a treatment option for glaucoma by restoring its function and lowering intraocular pressure.

These days the main treatment for glaucoma is to control the intraocular pressure to prevent vision loss. This is typically achieved through medication (eye drops), laser surgery and, sometimes conventional surgery.

The first treatment is eye drops. They work by either reducing the production of aqueous humor (fluid in the eye), by blocking receptors, inhibiting enzymes, or reducing nerve activity that stimulates fluid production. It is a treatment, not a cure.

Laser surgery helps to drain the aqueous humor by widening the natural drainage passages or creating new ones (hole in the riser to improve fluid flow). Laser surgery is not a final solution for glaucoma, but a valuable treatment. It can effectively lower intraocular pressure, but many patients still need medications and further treatments over time.

Conventional incisional surgery, like trabeculectomy, creates a new drainage path for fluid in the eye, or by using a tube shunt. As in the case of laser surgery, it is a treatment not a solution. It helps prevent any more damage, but it doesn't undo any damage that has occured; this is done as a last resort to stop any more eye damage.

As in the previous disease groups; your body knows what is wrong and how to fix it if the right stem cells are activated ; in this case they will be mesenchymal stem cells.

The mesenchymal stem cells derived from the adipose tissue or the trabecular meshwork itself. They can be induced to differentiate into functional trabecular meshwork cells, or any cell needed by the body to replenish the damaged tissue, create new drainage systems and anything required to improve the fluid drainage from the eye to return the drainage system to its original healthy state. The DNA Fixing Machine can activate the mesenchymal cells.

The orbital adipose tissue which has the mesenchymal stem cells is the fat tissue situated within the socket, cushioning and supporting the eyeball. Besides repairing the trabecular meshwork, the mesenchymal stem cells can alo repair the retina (the light-sensitive layer tissue at the back of the eye that can cause vision loss, blurred vision and night blindness); it can also repair the eye optic nerve (a sensory nerve that transmit visual information from the retina to the brain). If the optic nerve is damaged, loss of vision is the result.

Mesenchymal stem cells can also be used to reinforce the sclera (the outer layer of the eye), which will reduce or eliminate myopia symptoms.

In healthy eyes, light is able to pass through the lens back to the retina, allowing us to see detail. Cataract creates a cloud on the normally transparent lens, blurring vision and eventually leading to blindness. Essentially, the lens of the eye accumulates proteins and sugars throughout one's life and, ultimately this accumulation leads to blindness.

Cataracts are caused by the buildup of protein in the eye's lens, and these proteins are primarily produced by lens cells (specially, fiber cells). These fiber cells are packed with structural proteins called crystallins. When crystallin proteins clump together or degrade, they lose their transparency and scatter light, leading to the cloudiness of cataracts. The cost of treating cataracts in the USA is around 7 billion a year and it affects around 24.5 millions of Americans.

Cataracts are typically treated with cataract surgery, which involves removing the cloudy lens and replacing it with a clear, artificial intraocular lens. Approximately, 20% of the cataracts surgery patients need additional treatment, like capsulotomy.

The lens epithelial stem cells niche (another eye stem cell niche, there 3 different stem cell niches in the eye) is located on the anterior part of the lens capsule; it can generate new lenses to replace the old ones, ensuring the lens maintains its function. Studies had shown that the activation of the stem of the lens epithelial cells or LEs lead to the regeneration of a functional clear lens, eliminating the cataracts problem and the need for surgery. The DNA Fixing Machine is another option for cataracts removal, one done by your own body.

There are over 350 eye diseases that can be fixed by activating the correct eye stem cell niche and the DNA Fixing Machine can do this with no problem. The global economic productivity loss associated with vision impairment is 411 billion annually and over 28.5 billion in the USA alone.

### 8) Fixing of DNA Cell Mutations

There are two types of DNA mutation, one is called somatic mutations. They are a cell DNA change that occurs after fertilization, and it is not passed onto offspring. These arise in non-germline cells (cells that are not sperm or egg) and can occur in any body tissue. Cancer is the worst example of them; but cancer is such a different complicated disease that it has its own category.

These mutations can be caused by environmental factors, errors in DNA replication, and cell stress

A cell contains DNA in both the nucleus (where most of the DNA is located) and the mitochondria, which have their own small amount of DNA called mitochondrial DNA.

The nucleus is the computer program of the cell, so a DNA mutation there is essentially an instruction manual change that changes the cell functions. The mitochondrial are the power plants of the cells, some cells have one and other hundreds, depending on the cell needs; a DNA mutation on the mitochondria disrupt the ability of the cell to generate energy efficiency, which leads to diseases (most of the neuron cell mutations are due to changes on the mitochondria).

Most of the DNA mutations are irreversible, once a mutation occurs and it is accepted by the body as part of a normal cell, it will keep replicating. The cells have some limited repair mechanisms to return some mutated cells to their original state, but the best and fastest solution is to eliminate the mutated cells so new healthy ones are formed as replacement instead of trying to reverse the mutated cells to their original state.

There are thousands of somatic mutations, many of them have no effect and in some cases are beneficial. The treatment with the DNA Fixing Machine in the ones that need to be fixed is to kill the damaged mutated cells by 42 degrees heat waves, so healthy ones are formed as replacement.

Diabetics type II is an example of a somatic mutation that causes problem, the body's cells become resistant to insulin (see treatment of fat cells above, which is part one of the treatment), this caused the beta cells inside the pancreas to produce more insulin to compensate for this “body error”, or to overwork and this lead to cell stress.

Overworked beta cells, experienced chronic stress from high blood sugar levels that lead to cellular changes that are considered mutations, primarily due to the accumulation of damage between the cell, including DNA damage, which results in altered gene expression and dysfunctional production, impairing the cell' ability to produce insulin property. Also, this stress caused some beta cells to die, making the problem worse.

The first step in treating diabetes type II was explained above (fat cell treatment); the second part is to kill the mutated cells so new healthy ones can be produced instead.

In some cases it is recommended to activate stem cells to speed up the reproduction of stem cells to replenish the missing cells that were affected by somatic mutations due to age.

A good example of this are the melanin cells called melanocytes. Melanocytes are specialized cells that produce melanin, a pigment responsible for skin, hair and eye color, and also play a role in protecting against V radiation.

Everybody has the same number of melanocytes, but some make more melanin than others. If these cells make just a little melanin, your hair, skin, and yes can be very light. If your cells make more, then your hair, skin, and eyes will be darker.

There are two types of melanin: eumelanin (brown/black) and pheomelanin (red/yellow); the ratio of these two decides the skin, hair, and eye color. The ratio is mostly due to genetic factors.

As people age, the production of melanocytes gradually declines due to mutations and reduced stem cell activity, leading to a decrease in melanin production and the appearance of gray or white hair. As we age, the number and activity of stem cells that give rise to melanocytes (and hair follicles) diminish. These stem cells can become stuck in a non-functional state, unable to differentiate into melanocytes. Activating these stem cells with the DNA fixing Machine will return them to an active state.

Melanocyte stem cells reside in the bulge of hair follicles. This area, also known as the lower permanent portion of the hair follicle where the niche is located.

Birthmarks, age spots, etc. are caused by melanocytes malfunctions; replacing them by activating the stem cells will fix these problems, by also producing new healthy replacements.

The other type of DNA cell mutation are called germline mutations. There are over 8,000 genetic diseases caused by germline mutations. These mutations occur in the sperm or egg cells, meaning they can be passed down to future generations.

The DNA Fixing Machine cannot directly fix these diseases, because these mutations are part of the body, so the immune system treats them as “normal” healthy cells, but the DNA Fixing Machine can be used in combination with gene therapy to fix the problem.

Gene therapy is a very expensive treatment, the most expensive costing over 3.5 million dollars for a single treatment and normal ones costing between $60,000 to $630,000 for treatment. If the stem cells can be reproduced inside the body, this can reduce the treatment cost between 30% to 50%.

Gene therapy is a technique used to modify the stem cells by removing or fixing the damaged part, so when the stem cells reproduce, it will replace damaged cells with the correct ones. Cells have memory that is passed from generation to generation, so when a damaged cell is fixed even if it was part of the body from the beginning, the body knows it needs to be replaced.

Even in extreme cases like Down syndrome wherein 98% of the cases all the cells of the body has an extra chromosome 21, gene therapy is able to remove or silence the 21 chromosome, so if this is done at an early age and stem cell reproduction is activated by using the DNA Fixing Machine; this will create another treatment option.

When local temperature is used; the cell volume changes with temperature by following the volume expansion coefficient formula. This is what will happen when the DNA Fixing Machine is in operation; which is different to what happens when cells are mutated because their functions inside the cell changed and this affects their volume.

Mutations can affect the transport mechanisms directly affecting cell volume by regulating the movement of solutes (dissolved substances), across the cell membrane, which in turn influences the movement of water via osmosis, causing the cell to either swell or shrink (sometimes there is no change in volume), depending on the solute concentration gradient established by the transport process.

The cell volume expansion formula is:

$$Change in temperature = ( change in volume / ( initial volume * cell volume expansion ) )$$

Looking for 10% increase in volume for example, the local cell temperature will be:

$$T 2 - 37 = ( 0.1 V 1 / ( V 1 * 0.0003 ) ) = 296 degrees Celsius$$

Increasing or decreasing the local cell temperature for a few degrees as required by the DNA Fixing Machine won't affect the function of the healthy cells because their volume will be basically the same. Large changes in volume disrupt essential cellular functions (for example mutated cells in diabetics II are 10% smaller than similar healthy ones).

### 9) Killing of RNA and DNA Viruses

RNA viruses contain their genetic material as RNA (ribonucleic acid). Around 70% of the known viruses are RNA. They are the primary agents behind infectious diseases and epidemics in humans. Well known RNA viruses include influenza, HIV, Ebola, hepatitis C, polio, dengue, measles, Covi-19, etc.

Almost all of the RNA viruses are transmitted by ingestion, inhalation or direct contact. Once they are inside the body, they attach to specific receptors in host cells through proteins on their surface, then either fusing with the cell membrane (for enveloped viruses) or penetrating directly into the cell (for non-enveloped viruses), delivering their RNA genome inside the cell to initiate infection; this process usually occurs at the mucosal surfaces like the respiratory tack, digestive tract, or urogenital area depending on the virus type.

These viruses replicate in the cytoplasm of the cell (the gelatinous fluid that fills the inside of the cell), so they are outside the nucleus and the DNA. Some RNA viruses enter the nucleus like HIV and remain there as “dormant” viruses, causing a mutation. Dormant viruses will be another computer program or disease category.

RNA viruses are infectious particles that need a host cell to replicate.

RNA viruses weaken the cells by hijacking the cell's machinery to replicate their own viral RNA, essentially diverting the cell's resources away from its normal functions and often causing cellular damage. Damaged cells die at lower temperature than normal healthy cells because heat can further disrupt their already compromised structures. They die at temperatures around 40 degrees Celsius. This is the reason in some cases when the virus has replicated too much, your body temperature goes over 40 degrees Celsius to kill the infected cells, but we are not designed to cool the key organs if the whole body temperature is kept at above 40 degrees Celsius for too long.

RNA viruses also denature (modify their structure, so they can not reproduce anymore) at around 40 degrees Celsius, unfortunately the human body can not keep the whole body at 40 degrees for a long period of time, but the DNA Fixing Machine can do it at a local level.

Keeping the whole body to above 40 degrees for a long time can cause heat stroke damaging cells, tissues and organs.

RNA viruses can move to any cell, but generally they remain at specific areas, for example influenza and coronaviruses remain at respiratory epithelial cells, hepatitis C at the liver cells, etc. The ability to remain in a specific region will allow the DNA Fixing Machine to provide heat waves to that area to kill the infected cells, stopping the production of virus inside the body without damaging the healthy cells in that area.

The immune system is designed to kill RNA viruses, this is called acquired immune response, which is very slow to react. It takes days or weeks for the acquired immune response to be fully activated to fight the viruses.

There are some RNA viruses like the SARS-CoV-2 virus that causes the COVID-19 virus, that not only replicated very fast (they start massive replication in less than 48 hours), but they also trick the immune system to slow the immune response (they are called smart viruses); if you add this to persons that have weak or compromised immune system, the result is a deadly epidemic.

RNA viruses epidemics can be very deadly if not controlled, the influenza epidemic of 1,918 killed close to 50 million persons in the world. Having a machine that will destroy the factory of the RNA viruses inside your body is another tool in avoiding a future epidemic.

Even if there is not an epidemic, the yearly flu season is deadly for different reasons (weak immune system for example). Normally, the flu season can kill between 6,300 and 52,00 persons in the USA and between 290,000 and 650,000 persons in the world. The DNA Fixing Machine will work even if your immune system is weak.

Another example of dangerous RNA virus is the HIV virus, there are HIV medications that work by blocking a protein the infected cells need to make new HIV copies, but this is not a cure. During the infection period, the HIV virus can be found on the thymus contaminating the production of the CD4+T immune cells and weakening the immune system.

Right now vaccines are the most used treatment for RNA viruses, but once the virus mutated, which RNA virus do very often, you will need a new vaccine and from this came the name monoclonal antibodies, mono is one, clone is an inactivated virus, nucleic acid or virus like particle and antibody is the protein produced by the immune system that recognizes and neutralizes specific foreign substances called antigens. For each antigen, there is only one antibody produced.

The idea of the vaccine is that the immune system already knows the virus, so when it enters the body the immune system reaction will occur right away instead of taking days or weeks to fight the RNA viruses.

Medical staff tried to reduce the mutation process by vaccinating as many people as possible, to eliminate the change of the virus from moving from person to person, especially if a weak but alive virus is used as a vaccine.

mRNA vaccines don't need to use a live virus to simulate disease-fighting response, they carried genetic instructions to activate an immune response, enabling your immune system to combat the actual virus should you be exposed to it, but some vaccines like measles or polio use weak live virus to produce a stronger response that will give you protection for live or for many years.

During infection, temperature rises systemically (as fever) because the body's immune system releases chemicals called pyrogens that signal the hypothalamus (the brain's temperature control center) to raise the body's temperature set point, not just at the site of the infection, which will be a better solution.

RNA viruses affect organelles inside the cell, including mitochondria (power plan of the cell), golgi complex (manufacturing and processing of cell proteins), endoplasmic reticulum (highways inside the cell), etc.

The other type of viruses are the DNA viruses, they have a genome (a set of DNA instructions, composed of deoxyribonucleic acid. They duplicate within the nucleus of the infected cell.

The most dangerous DNA virus is the variola virus, which causes smallpox. In the 20th century smallpox killed between 300 and 500 million people in the world.

As in the case of MRA virus, vaccines are used to prepare the immune system for DNA viruses.

There are other notable DNA viruses like the Herpes simplex virus (causes cold sores and can lead to serious complications in certain individuals), Human papillomavirus (linked to cervical and other cancers), Adenovirus (can cause respiratory infections, including the common cold), Epstein-Barr virus (associated with infectious mononucleosis and certain cancers).

The main differences between DNA vaccines and mRNA is that the DNA exists naturally, while mRNA is made from DNA in a process called transcription. The DNA vaccines can be stored at room temperature for months, mRNA vaccines must be handled carefully and kept very cold during transportation and storage, and they can only be removed from cold storage shortly before they are administered.

Very few DNA vaccines are approved for use outside of clinical trials and veterinary purposes because the DNA vaccines could alter the DNA sequence of the cell.

As in the case of RNA viruses, DNA viruses denature (stop production at 40 degrees Celsius) and the infected cells will also die at 40 Celsius (weakened cell).

The DNA Fixing Machine can be used to kill DNA viruses inside the body if needed by keeping the area where the infected cells are located at 40 degrees Celsius., eliminating the production of DNA viruses inside your body is needed.

DNA viruses are passed from person to person through direct contact with bodily fluids, coughing, sneezing, contact with contaminated surfaces, etc.

Another big difference between RNA and DNA viruses is that DNA viruses are most stable (as any case with DNA, they have proofreading mechanisms that stop a lot of mutations during duplication), so they mutated at a lower rate compared to RNA viruses, meaning that if there is a vaccine for a DNA virus, there is a good change that no new vaccines will be needed for that disease for a long time.

### 10) Microglia—Brain Immune System

One the treatment of neuron diseases is to reproduce the missing cells to fill the holes in the brain. The other part is to remove the plaques that caused these holes.

In the case of Parkinson's disease, the brain cells that died are dopamine neurons located in the midbrain. They died because of their accumulation of a misfolded protein called alpha-synuclein which forms toxic clamps or plaques within the cells, disrupting their function and eventually leading to cell death; this loss of dopamine, a crucial neurotransmitter for movement control results in the motor symptoms of Parkinson's disease.

There are other dangerous diseases caused by plaques on the brain such as Alzheimer's disease, Dementia, Frontotemporal dementia, Cerebral amyloid angiopathy (it can lead to paralysis, and even coma), etc.

For a complete cure of neurological disorders, removing the toxic clamps or plaques that caused these diseases is a requirement, otherwise you will be needing to reproduce neuron cells over and over again; it will be a treatment not a cure.

The best way to remove the toxic clamps inside the brain is by activating the brain's natural cleaning system, the microglia. They are the immune cells in the brain.

Migroglia are resident immune cells in the central nervous system (CNS) including the brain and spinal cord. They play crucial roles in maintaining brain homeostasis, responding to infections and injuries, and regulating brain development and function. Migrolia in the spinal cord reduces inflammation, mitigates oxide stress and helps repair damaged nerve tissue.

Migroglia in the brain continuously monitor the brain for pathogens, damaged cells and other threats. They engulf and remove debris, including dead cells, bacteria, and viruses. Microglia respond to infections and injuries by releasing inflammatory mediators, such as cytokines (communication proteins) and chemokines (proteins that regulate immune system migration).

Migroglia in some cases release protective factors that help neurons survive. They also contribute to the formation and maintenance of synapses (places where neurons connect and communicate with each other); they also maintain the balance of fluids, ions, and nutrients in the brain.

Migroglia is activated by temperature. This is one of the reasons why during a flu your temperature goes up; the body wants to protect the brain by activating the microglia by the thermosensitive ion channel TRPV4, which allow microglia to detect and respond to fluctuations in the body temperature within the physiological range.

Migroglia is activated at around 39 or 40 degrees Celsius.

The solution to remove toxic plaques in the brain is by activating Microglia. Migroglia is not a stem cell, so it is activated by using a “soft” start process.

The DNA Fixing Machine can be used to activate the microglia niche, it is found in the subventricular zone of the brain.

Microglia can clear plaques including the ones that produce Alzheimers by different mechanisms, including engulfing and digesting them, and releasing digestive enzymes.

As we age, microglia undergo significant changes. They become more activated and lose the ability to perform essentially functions like debris clearance and immune surveillance.

Activating microglia from the niche by a heat shock will produce a different response that activates microglia by age. Activation from the niche will respond as the activation was done by brain injury, an infection, a neuroinflammation or a neurodegeneration, so the activated cells will do the cleaning job they lose due to age; activating Migroglia by a heat shock will rejuvenate the cells.

### 11) Eliminate Dormant Viruses and Stop Long Term Effects

Dormant viruses are RNA or DNA viruses that remain inactive within the body after an initial infection, without causing any noticeable symptoms. They persist in a hidden state, known as latency and they can remain inactive within the body of an infected host without causing any noticeable symptoms or disease. They can be priests for extended periods, sometimes years or even decades. They hide in the DNA or nucleus of cells.

For example, herpes simplex viruses hide in the trigeminal ganglion, a group of nerve cells at the base of the brain.

Varicella zoster virus (chicken pox) can remain dormant in the spinal cord's nerve cells. It can “wake up” later on (when people are over 50 years old); that is the reason they are shingles vaccines to “charge” the immune system for this virus.

Another good example of a dormant virus is ebola (which has a 50% fatality rate), it can remain dormant on the testicles, nervous system, and interior of the eyes.

In long term effects (called chronic diseases because it is a long term condition that can not be cured), people report having health problems long after having the disease, a good example is Covi-19. There are many theories for this, but one of the most popular is that Covi-19 virus hides in the gut cells and from there it spreads to other parts to cause health issues.

Dormant viruses are linked to many other diseases, including birth defects (Zika virus). They are reactivated during pregnancy and transmitted to the fetus, leading to conditions like microcephaly, a condition where a baby's head is significantly smaller than expected, often due to abnormal brain development. The consequences of this are children with delay in reaching developmental milestones, and many of them have intellectual disabilities.

Dormant viruses can also cause hearing loss, abnormal calcium deposits in the brain, slowed fetal growth, enlarged liver or spleen, poor eyesight, etc.

The medical field has an idea of the area where each dormant virus hides; but to treat them, the virus needs to be activated.

The majority of the dormant viruses are activated by cold temperatures and others by hot temperatures.

The DNA Fixing Machine can do both, activate the virus by cold temperature and later kill them by hot temperature and it can do this on the same treatment session. The DNA Fixing Machine has the ability to provide cold waves at the temperature needed and change in a matter of minutes to provide heat waves without removing the insert wire into the patient.

Dormant viruses, once activated, will weaken the cell and the affected cells will die at 42 degrees Celsius.

Dormant viruses are treated by addressing their reactivation and supporting the body's immune response; there is no active cure for this, so it is an open field for the DNA Fixing Machine.

The DNA Fixing Machine doesn't have compressors and the only moving mechanical parts are the two small fans; it is very quiet and it can be used in medical places without modifications. It is designed to go through a normal 3 feet door and it will last 40 years with no or little maintenance.

### 12) Fix Hormone Imbalances

A hormonal imbalance happens when you have too much or too little of one or more hormones—your body's chemical messengers, concentrated in a group of the same cell type.

Some of the most common hormonal imbalance diseases are infertility (low testosterone levels in men causes infertility and hormonal imbalance disrupt ovulation in females making it impossible to conceive), it can also contribute to obesity.

There are cosmetic diseases caused by hormone imbalance like missing hair; fluctuations in hormones like estrogen, progesterone, and androgens lead first to hair thinning and later to missing hair.

Hormones directly impact the hair growth cycle, influencing the length of time hair follicles stay in the growth phase, they also shrink hair follicles causing missing hair on the scalp.

Other cosmetic problems caused by hormone imbalance are wrinkles, age spots, and other problems found in the three layers of the skin. As hormone levels change with age, skin becomes thinner, drier, less elastic which leads to wrinkles.

Low estrogen levels besides causing wrinkles can also cause sagging skin. Estrogen helps maintain skin thickness and collagen production. Hormone changes, particularly in estrogen and progesterone can increase melanin production, this lead to hyperpigmentation and age spots.

One of the most dangerous hormonal imbalance diseases include acromegaly, this results from the overproduction of growth hormone, leading to abnormal growth of bones, organs, and tissues. The opposite of this is a mutation on the pituitary gland that produces insufficient growth hormone, it can lead to growth hormone deficiency (GHD), also known as pituitary dwarfism, characterized by abnormally short stature.

The body controls hormone production through a system called “negative feedback loop”, where the level of hormones in the bloodstream signals the hypothalamus gland to increase or decrease its production, essentially maintaining a balanced level of that hormone within the body. The hypothalamus in the brain acts as the primary control center, sending signals to the pituitary gland which then regulate other endocrine glands to release hormones as needed.

The hypothalamus is located in the lower part of the brain, and acts as a bridge between the nervous and endocrine systems. It receives information from the brain and sends signals to the pituitary gland, influencing the release of hormones.

The pituitary gland situated at the base of the brain, produces hormones that control many other endocrine glands.

Hormonal imbalance can be the result of faulty hypothalamus or a faulty pituitary gland.

The human body makes over 50 different hormones. Hormones connect to the organs and tissues by acting as chemical messengers, traveling through the bloodstream to reach specific target cells, where they bind to receptors and trigger specific responses based on the hormone's signal.

A faulty hypothalamus can produce hormone imbalances by either over or under secreting releasing hormones, which signal the pituitary gland to adjust the production of other hormones in the body, leading to imbalances in the levels of those hormones depending on which releasing hormone is affected; if the hypothalamus malfunction, it disrupts the normal feedback loop that regulates hormone levels throughout the body.

A faulty pituitary gland can affect the hormone balance by interpreting the wrong signal from the hypothalamus.

Medical research and studies suggest that the tancyte stem cells in the hypothalamus can repair damaged hypothalamus.

They will differentiate to the required cell needed by the hypothalamus to fix the damage.

The tancyte cell niche inside the hypothalamus is located in the third ventricle.

The pituitary stem cell niche is found inside the pituitary gland. They can differentiate in any type of cell needed by the pituitary gland to fix any cell mutation.

Most of the hormone imbalance is due to a faulty hypothalamus.

To find if a hormone imbalance is due to the hypothalamus or the pituitary gland, careful diagnostic processes are required involving a combination of physical examinations, medical history, and laboratory tests.

Hypothalamus is the thermostat of the body; the tancyte stem cells located inside the hypothalamus are activated at 40 degrees Celsius, if the temperature is kept for an hour. Being the hypothalamus the thermostat of the body, it is important only to increase the temperature of tancyte stem cells; something that the DNA Fixing Machine can do.

If the whole hypothalamus is kept at 40 degrees Celsius; this could lead to cell protein damage, cell death, potential organ damage, and even brain damage.

The activation of the pituitary stem cell niche is not done by temperature, it is activated by injury. Injury to the pituitary gland is done by endocrine cell ablation (remove surface tissue) these days, the cost of this is around $35,000.

The DNA Fixing Machine can be used to damage a tiny part of the pituitary gland, the damaged part area and its dimensions can be selected by the medical facility to activate the pituitary stem cell niche. The stem cells of the pituitary gland don't need a specific temperature to function, so as soon as a single cell at the surface of the pituitary gland is damaged, the stem cell production will start and any damaged previous cell will be fixed.

Any temperature above 39 degrees Celsius will damage pituitary glands, it is not recommended to go over 41 degrees Celsius because more significant damage will occur; the idea is to slightly damage the surface cells to activate the stem cell niche but not to case any significant damage to pituitary gland that will take more time to fix. The DNA Fixing Machine can do this.

Most hormone imbalances are treated with medications these days, in the majority of the cases, once the balance is restored, no medications will be required.

The ideal use of a DNA Fixing Machine will be for chronic hormone imbalance cases where medication won't work.

### BRIEF DESCRIPTION OF DRAWINGS

The provided drawings show all the parts needed to build the DNA Fixing Machine. They all can be made by using existing companies.

1. The DNA Fixing Machine can cure cancer
it does it in 3 different ways
A) It can kill cancer cells by using hot temperatures.
B) It can kill cancer cells by returning them to its original cell volume.
C) It can kill cancer cells by using cold temperatures.

2. The DNA Fixing Machine can reproduce neuron cells
It does it by
activating the neural stem cell niche.

3. The DNA Fixing Machine can provide blood vessel maintenance
it returns blood flow to laminar by
heating the blood and using a stent.

4. The DNA Fixing Machine can fix the Thymus organ
it activates the Poly Keratin stem cells,
their reproduction replaces missing or damaged cells in the Thymus.

5. The DNA Fixing Machine can remove visceral fat cells
it does it by
cooling the visceral fat cells inside the body.

6. The DNA Fixing Machine can regenerate tissues and organs
it does it by
the removing scar tissue, creating a blastema and activating the Pluripotent stem cells.

7. The DNA Fixing Machine can cure eye diseases
it does it by
activating the 3 different stem cell niches in the eye.

8. The DNA Fixing Machine can fix both types of DNA mutations
somatic mutations can be fixed by killing the mutated cells
Germline mutations can be fixed by working with gene therapy.

9. The DNA Fixing Machine can kill RNA and DNA viruses
it does it by
killing the contaminated cells inside the body by heat waves.

10. The DNA Fixing Machine can activate the brain immune system
it does it by
activating the Migroglia cells inside the brain.

11. The DNA Fixing Machine can eliminate dormant viruses and stop long term effects
it does it by
activating the dormant viruses and later by killing them with heat waves.

12. The DNA Fixing Machine can fix hormone imbalances
it does it by
activating the tancyte cell niche inside the hypothalamus and the pituitary stem cell niche inside the pituitary gland.
